(19) United States
(12) Patent Application Publication
(10) Pub. No.: US 2025/0064438 A1
(43) Pub. Date: Feb. 27, 2025

(54) SYSTEM AND METHOD FOR FASTENING A TUBULAR PROSTHESIS

(71) Applicant: THE PENN STATE RESEARCH FOUNDATION, University Park, PA (US)

(72) Inventors: Robert DOWLING, University Park, PA (US); William S. PIERCE, University Park, PA (US); Gerson ROSENBERG, University Park, PA (US); William J. WEISS, University Park, PA (US)

(21) Appl. No.: 18/721,118

(22) PCT Filed: Dec. 16, 2022
(86) PCT No.: PCT/US22/53198

§ 371 Date: Jun. 17, 2024

Related U.S. Application Data
(60) Provisional application No. 63/291,049, filed on Dec. 17, 2021.

(51) Int. Cl. A61B 17/00; A61B 17/11

(52) U.S. Cl. CPC A61B 17/00; A61B 2017/1107; A61B 2017/1132

(57) ABSTRACT

A prosthetic anastomosis fixation device comprising an inner sleeve sized and configured to be received within a blood vessel of a patient, the inner sleeve including a projection (e.g., pins) extending from an outer surface of the inner sleeve and a groove provide on the outer surface spaced apart from the projection, and a fixation ring removably received over the inner sleeve, wherein engagement between the projection and the fixation ring couples the fixation ring to the inner sleeve.

### CROSS-REFERENCE TO RELATED APPLICATIONS

This application claims the benefit of U.S. Provisional Application No. 63/291,049 filed, Dec. 17, 2021, the contents of which are incorporated herein by reference in their entirety.

### TECHNICAL FIELD

This disclosure relates to anastomosis devices for connecting native blood vessels to vascular grafts and other prosthetic devices.

### BACKGROUND

The resection and replacement of the aorta for aneurysmal disease and dissection is a common operation in which the use of handsewn anastomosis to connect vascular grafts to native blood vessels remains the standard of care. In the dissection of the ascending aorta, replacement is required as the mortality with non-operative therapy is very high (1% mortality/hour). Though the exact incidence of replacement of the aorta for dissection is not known population-based studies suggest an incidence in the US between 30,000 to 80,000 cases annually. The dissection disrupts the normal tissue integrity and makes the creation of standard handsewn anastomosis among the most difficult procedures in cardiac surgery. As a result, time to create this anastomosis is often long and suture line bleeding is common and can be very difficult to manage. Additionally, these operations require a period of hypothermic circulatory arrest to perform the distal aortic anastomosis. The time to create the anastomosis often results in significant periods of circulatory arrest with longer periods of circulatory arrest being associated with increased neurologic events and end-organ dysfunction.

Elective resection of aortic aneurysms is also a common operation in which the use of handsewn anastomosis is typically used. The exact number of elective resections of aortic aneurysms is unknown but estimates from the Society of Thoracic Surgeons suggest between 15,000 and 20,000 cases per year in the US. Likely a similar number of cases are performed in the EU. These patients often have thin-walled aortas and require periods of hypothermic circulatory arrest. Creating a fast, hemostatic anastomosis with a device as described herein that eliminates the use of needles and sutures will prevent issues related to fragile native tissue and abrogate issue related to prolonged times to create an anastomosis. Moreover, this approach could potentially improve patient outcomes by allowing surgeons to safely increase the extent of aortic resection. Up to 30% of patients that have resection of their ascending aorta will require an intervention for progressive aneurysmal disease of their aortic arch. A rapid and effective anastomotic device as described herein will allow surgeons to extend the resection to include the aortic arch followed by a rapid secure anastomosis to the proximal descending thoracic aorta. Rapid anastomoses to the arch branch vessels can then be performed eliminating concerns for progression of aortic arch disease.

Heart failure is another example procedure where a handsewn anastomosis is used to connect the native atrium to the artificial heart. The incidence of heart failure continues to increase while the prognosis remains poor, and few options are available for patients who fail medical therapy. Heart transplantation is limited to fewer than 6,000 procedures per year globally. The National Institutes of Health continues to identify the need for improved mechanical circulatory support (MCS) devices and has estimated that up to 175,000 patients could immediately benefit from MCS. Current generation left ventricular assist devices (LVADs) have improved outcomes but are still associated with significant morbidity a including a high rate of stroke (8% at 1 year) and mortality (5 year survival of 46%). Importantly, continuous flow total artificial hearts (TAH) has been developed, however, a major issue related TAH implantation is the extent of the operative therapy. Creation of the anastomoses of the native atrium to the atrial cuff of the TAH is particularly challenging due to thin atrial tissue with frequent tears of the tissue and/or needle hole bleeding. A double suture line, which is time consuming, is often used to avoid both bleeding and air entrainment through the suture line with the potential for cerebral air emboli. Development of a TAH atrial cuff, as described herein, that can be rapidly anastomosed to the native atrial tissue and provides improved hemostasis will: (1) decrease the complexity of the operation, (2) decrease intraoperative and perioperative bleeding and, (3) decrease the time on cardiopulmonary bypass. Longer times on cardiopulmonary bypass increase both operative morbidity and mortality.

In summary, a need in the art exists for a quickly and easily implanted, sutureless anastomosis for connecting native blood vessels to vascular grafts, total artificial heart devices, and/or other biological conduit such as a bile duct, ureter, and/or fallopian tube.

### SUMMARY

Certain examples of the present disclosure provide sutureless anastomosis fixation device.

A prosthetic anastomosis fixation device disclosed herein comprises an inner sleeve sized and configured to be received within a blood vessel of a patient, the inner sleeve including a projection (e.g., pins) extending from an outer surface of the inner sleeve and a groove provide on the outer surface spaced apart from the projection; a fixation ring removably received over the inner sleeve; wherein engagement between the projection and the fixation ring couples the fixation ring to the inner sleeve.

A method of attaching a prosthetic anastomosis device to a patient's blood vessel using a fixation device disclosed herein comprises: advancing the proximal end of an inner sleeve within a blood vessel of a patient, the inner sleeve including pins extending from an outer surface of the inner sleeve; securing the inner sleeve to the blood vessel by engagement between the pins and the blood vessel; providing a fixation ring around the inner sleeve such that the blood vessel is positioned between the inner sleeve and the fixation ring around a circumference of the inner sleeve, and at least one pin extends through the blood vessel toward the fixation ring; advancing a second tubular component (e.g., prosthetic device) on the distal end of the inner sleeve; providing a ligature around the inner sleeve such that the second tubular component is provided between the inner sleeve and the ligature around the circumference of the inner sleeve; tightening the ligature around the inner sleeve and the second tubular component, wherein the radial inward force provided by the fixation ring creates a liquid-tight seal between the outer surface of the inner sleeve and the blood vessel, and wherein tightening the ligature around the inner sleeve and the second tubular component creates a liquid-tight seal between the outer surface of the inner sleeve and the second tubular component.

A further implementation of a prosthetic anastomosis fixation device disclosed herein comprises: an inner sleeve sized and configured to be received within a blood vessel of a patient, the inner sleeve including pins extending from an outer surface of the inner sleeve; a tubular component (e.g., prosthetic device) provided within a central lumen of the inner sleeve, a proximal end of the tubular component extending beyond a proximal end of the inner sleeve and a portion of the tubular component folded over the proximal end of the inner sleeve; wherein engagement between the folded over portion of the tubular component and the pins couples the tubular component to the inner sleeve.

A further method of attaching a prosthetic anastomosis device to a patient's blood vessel using a fixation device disclosed herein comprises: advancing a proximal end of a tubular component (e.g., prosthetic device) through a central lumen of an inner sleeve; advancing the proximal end of the tubular component beyond a proximal end of the inner sleeve; folding a portion of the tubular component over the proximal end of the inner sleeve such that the folded portion of the tubular component extend along an outer surface of the inner sleeve; coupling the tubular component to pins projecting from the outer surface of the inner sleeve; providing a fixation ring around the inner sleeve and the tubular component such that the fixation ring extends over the pins; wherein the radial inward force provided by the fixation ring creates a liquid-tight seal between the tubular component and the inner sleeve.

Another implementation of a prosthetic anastomosis fixation device disclosed herein comprises a sleeve-style atrial connector including: a flared sleeve; a circular band coupled to a proximal end of the flared sleeve, the band including pins projecting radially outward from an outer surface of the band; a fixation ring received over the band such that the pins extend at least partially into the fixation ring; wherein the radially inward force provided by the fixation ring creates a liquid-tight seal between the band and the fixation ring.

Another method of attaching a prosthetic anastomosis device to a patient's blood vessel using a fixation device disclosed herein comprises: advancing a blood vessel on a proximal end of a prosthetic fixation device, the device including a flared sleeve and a circular band coupled to the distal end of the flared sleeve, the band including pins projecting radially outward from an outer surface of the band; coupling the blood vessel to the pins projecting from the band; providing a fixation ring around the flared sleeve and the band such the fixation ring extends over the pins and the blood vessel is positioned between the fixation ring and the band; securing the fixation ring around the flared sleeve and the band; wherein the radial inward force provided by the fixation ring creates a liquid-tight seal between the tubular component and the inner sleeve.

A further implementation of a prosthetic anastomosis fixation device disclosed herein comprises a sheet for joining adjacent tissue segments, the sheet comprising: a flexible substrate including at a plurality of protrusions extending from an inner surface of the substrate, wherein the protrusions are sized and configured to anchor the substrate to a tissue of a patient.

A further method of attaching a prosthetic anastomosis device to a patient's blood vessel using a fixation device disclosed herein comprises: positioning a flexible substrate adjacent a first tissue segment, the substrate including a first plurality of protrusions extending from an inner surface of the substrate, the protrusions sized and configured to anchor the substrate to a tissue of a patient; fixing the first plurality of the protrusions to the first tissue segment; positioning the flexible substrate adjacent a second tissue segment; and fixing a second plurality of the protrusions to the second tissue segment.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A is a schematic drawing of a patient anatomy with a prosthetic fixation device according to some examples.

FIG. 1B is a schematic drawing of a patient anatomy with a prosthetic fixation device according to some examples.

FIG. 2 is a side cross-section view of the example inner sleeve of FIGS. 1A and 1B.

FIG. 3 is an end view of the inner sleeve of FIG. 2.

FIG. 4 is an end view of the example fixation ring of FIGS. 1A and 1B.

FIG. 5 is a side view of the fixation ring of FIG. 4.

FIG. 6 is a perspective view of the fixation ring of FIG. 4.

FIG. 7 is a schematic drawing of a patient anatomy with a prosthetic fixation device according to some examples.

FIG. 8 schematic drawing of a prosthetic fixation device according to some examples.

FIG. 9 is a schematic drawing of the fixation device of FIG. 9 including corresponding patent anatomy.

FIG. 10 is a schematic drawing of the fixation device of FIG. 9 including corresponding patent anatomy.

FIG. 11 is a cross section view of the fixation device of FIG. 9 including corresponding patent anatomy.

FIG. 12 is a partial cross section view of the fixation device of FIG. 9 including corresponding patent anatomy.

FIG. 13 is a top perspective view of the fixation device of FIG. 9 attached to a holder.

FIG. 14 schematic drawing of a prosthetic fixation device according to some examples.

Like reference symbols in the various drawings indicate like elements.

### DETAILED DESCRIPTION

Disclosed herein is a rapid, sutureless anastomosis that provides significant advantages over current handsewn techniques with the goal to improve patient outcomes. The present disclosure provides for devices and methods that allow for a rapid and hemostatic sutureless anastomoses in both cardiac and vascular surgery and other procedures involving the attachment of grafts or other materials to hollow viscus such as urologic or intestinal procedures. For example, the disclosed device and method can be used to provide anastomoses of grafts to the native heart, native blood vessels or any hollow viscus and can also benefit patients that require placement of a total artificial heart. As described herein, the anastomotic fixation device rapidly creates an anastomosis without any tissue penetration through the native tissue in a method that is much quicker compared to traditional handsewn techniques. Moreover, the elimination of needle hole bleeding and decreased operative times will lead to improved patient outcomes.

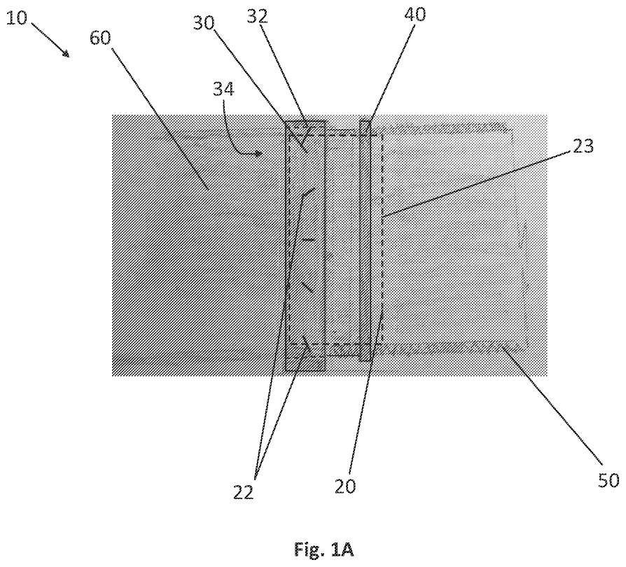

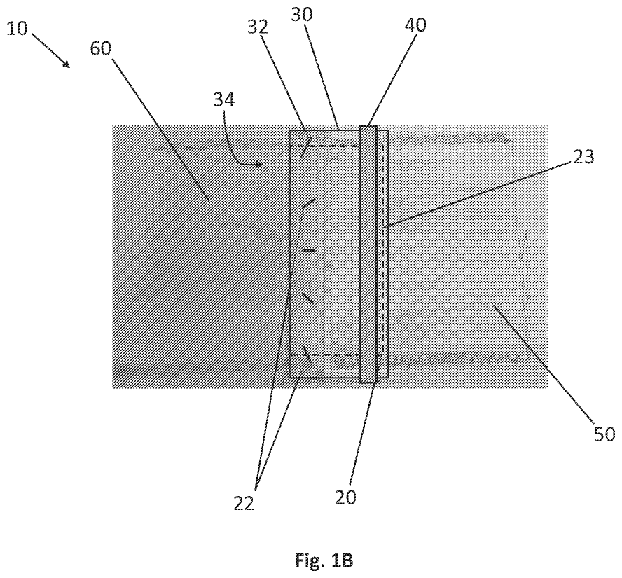

FIGS. 1A and 1B illustrate a schematic drawing of a patient anatomy with an example prosthetic anastomosis fixation device 10. The fixation device 10 includes an inner sleeve 20 and a fixation ring 30 removably received over the inner sleeve 20. The inner sleeve 20 is sized and configured to be received within portion of a patient's anatomy 60 (e.g., blood vessel). The inner sleeve 20 includes at least one projection 22 extending from an outer surface 24 of the inner sleeve 20 and configured to extend into/through the patient anatomy 60. The fixation ring 30 is received over the projection 22 such that the fixation ring 30 is coupled to the inner sleeve 20 and a portion of a patient's anatomy 60 is fixedly secured between the inner surface 32 fixation ring 30 and an outer surface 24 of the inner sleeve 20. For example, the patient's blood vessel is secured circumferentially between the inner sleeve 20 and the fixation ring 30. A prosthetic device 50 is coupled to the inner sleeve 20 and/or the fixation ring 30. Example prosthetic devices 50 include a graft material and/or other biological conduit. As a result, the prosthetic device 50 quickly and easily couples segments of vascular tissue/biological conduits, e.g., diseased segments of vascular tissue or other biological conduit).

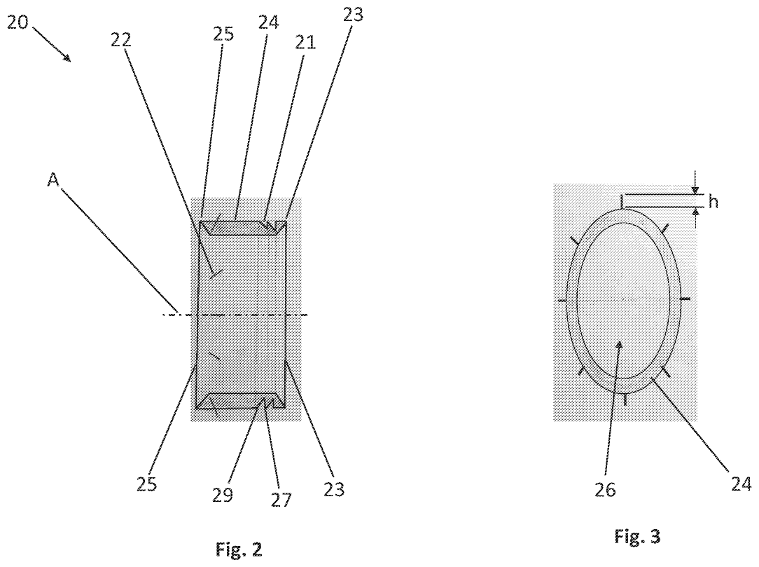

FIGS. 2 and 3 provide a side cross-section and end view of the example inner sleeve 20 of FIGS. 1A and 1B. The inner sleeve 20 defines an annular generally cylindrical-shaped structure with a central lumen 26 extending therethrough. As illustrated in FIGS. 1A and 1B, the inner sleeve 20 (or outer fixation ring 30) is coupled to the prosthetic device 50 at is proximal ends 25.

As provided in FIGS. 1A and 1B, the inner sleeve 20 is sized and configured to fit snugly on the inside of the patient anatomy 60 (e.g., inner surface of the blood vessel). That is, the inner sleeve 20 has an outer diameter corresponding to the inner diameter of the target patient anatomy 60 including, for example, blood vessels and any other biological conduit such as a bile duct, ureter, or fallopian tube. For example, the outer diameter of the inner sleeve 20 ranges from 1 mm to 100 mm, including exemplary values of 1 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm. In still further aspects, the diameter can have any value between any of the foregoing values. For example, the diameter can be between (and including) 3 mm to 35 mm, 10 mm to 50 mm, 10 mm to 30 mm, 18 mm to 36 mm, 50 mm to 55 mm, 50 mm to 100 mm.

As provided in FIG. 1A, the axial length of the inner sleeve 20 is greater than the axial length of the fixation ring 30. In some examples, the length of the inner sleeve 20 ranges from 12 mm to 45 mm, including exemplary values of 18 mm, 20 mm, 21 mm, 22 mm. In still further aspects, the length of the inner sleeve 20 can have any value between any of the forgoing values. For example, the length can be between (and including) 20 mm and 21 mm. In other examples, as provided in FIG. 1B, the axial length of the inner sleeve corresponds to or is great than the axial length of the fixation ring 30.

The wall thickness of the inner sleeve 20 ranges from 0.5 mm to 3.5 mm, including exemplary values of 0.5 mm, 1.0 mm, 1.5 mm, 1.57 mm (0.062 inches), 2.0 mm, 2.5 mm, 3.0 mm, 3.18 mm (0.125 inches), 3.5 mm. In still further aspects, the wall thickness of the inner sleeve 20 can have any value between any of the forgoing values. For example, the wall thickness can be between (and including) 1.57 mm and 3.18 mm

The inner sleeve 20 includes at least one projection 22 extending radially from the outer surface 26 of the inner sleeve 20. In some examples, the projection 22 includes a plurality of pins 22 extending radially from the outer surface 24 of the inner sleeve 20. For example the inner sleeve 20 includes a number of pins 22 ranging from 1 to 30 pins, including exemplary values of 1 pin, 2 pins, 3 pins, 4 pins, 5 pins, 6 pins, 7 pins, 8 pins, 9, pins, 10 pins, 11 pins, 12 pins, 13 pins, 14 pins, 15 pins, 16 pins, 17 pins, 18 pins, 19 pins, 20 pins, 21 pins, 22 pins, 23 pins, 24 pins, 25 pins, 26 pins, 27 pins, 28 pins, 29 pins, 30 pins. In further examples, the inner sleeve 20 includes a number of pins 22 ranging from 4 to 8 pins.

The pins 22 have a sharpened distal tip for engaging and/or passing through the patient anatomy 60. As illustrated in FIGS. 1A, 1B and 2, the pins 22 extend in a direction transverse to a longitudinal axis A of the inner sleeve 20. For example, the pins 22 extends in a direction towards a distal end 23 of the inner sleeve 20. In other examples, the pins 22 extend perpendicular to the longitudinal axis A of the inner sleeve 20. In further examples, the pins 22 extend in a direction towards the proximal end of the inner sleeve 20. The angle of the pins 22 with respect to the longitudinal axis A ranges from 1° to 90°. As provided in FIGS. 1A, 1B and 2, each of the plurality of pins 22 extend in the same direction. In other directions, each of the pins 22 extend in varied and/or alternating directions.

The pins 22 are equally spaced circumferentially around the outer surface 24 of the inner sleeve 20. In further examples, the pins 22 are asymmetrically spaced around the circumference of the inner sleeve 20.

The pins 22 have a diameter ranging from 0.3 mm to 1.0 mm, including exemplary values of 0.1 mm, 0.2 mm, 0.3 mm, 0.4 mm, 0.5 mm, 0.6 mm, 0.7 mm, 0.8 mm, 0.9 mm, 1.0 mm. As illustrated in FIGS. 1A and 1B, the height of the pins 22 is less than a wall thickness of the fixation ring 30 such that the pins 22 extend through a portion of a wall thickness of the fixation ring 30. The height (h) is measured radially from the outer surface 24 of the inner sleeve 20 and the distal end of the pin 22, in a direction perpendicular to the longitudinal axis A of the inner sleeve 20. In other examples, the height of the pins 22 is greater than a wall thickness of the fixation ring 30 such that the pins 22 extend through an entire wall thickness of the fixation ring 30 and beyond the outer surface of the fixation ring 30. The height of the pins 22 ranges from 1.5 mm to 5 mm, including exemplary values of 1.5 mm, 2.0 mm, 2.5 mm, 3.0 mm, 3.5 mm, 4.0 mm, 4.5 mm, 5 mm. The length of the pins 22 (measured axially along the pin 22) ranges from 1 mm to 4 mm, including exemplary values of 1.0 mm, 1.5 mm, 2.0 mm, 2.5 mm, 3.0 mm, 3.5 mm, 4.0 mm.

As illustrated in FIGS. 1A, 1B and 2, the inner sleeve 20 includes a groove 21 on the outer surface 24 spaced apart from the pin 22. The inner sleeve 20 can include a single groove 21 or a plurality of grooves 21 on the outer surface 24. The groove 21 extends at least partially around the circumference of the inner sleeve 20.

The pins 22 are provided adjacent a proximal end 25 of the inner sleeve 20 and the groove 21 is provided adjacent the distal end 23 of the inner sleeve 20 opposite from the pins 22. In some examples, the groove 21 includes rectilinear or V-shaped groove 21 extending circumferentially around the inner sleeve 20. For example, the groove includes a V-shaped groove 21 defined by a shoulder 27 and a tapered surface 29 extending from an inner edge of the shoulder 27 toward the proximal end 25 of the inner sleeve 20. In further examples, the groove 22 includes a curved or U-shaped recess extending circumferentially around the inner sleeve 20.

As described above, the fixation device 10 includes a fixation ring 30 positioned circumferentially around the inner sleeve 20. As illustrated in FIGS. 1A, 1B and FIGS. 4-6, the fixation ring 30 defines a generally annular ring-shaped structure with a central lumen 34 extending therethrough. The fixation ring 30 is positioned around the outer surface of the inner sleeve 20 with the patient anatomy 60 therebetween. The pins 22 secure the position of the inner sleeve 20 with respect to the patient anatomy 60 and the radially inward force provided by the fixation ring 30 creates a liquid-tight seal between the inner sleeve 20, fixation ring 30 and patient anatomy 60. The fixation ring 30 has an inner diameter less than an outer diameter of the inner sleeve 20 such that the fixation ring 30 is stretched radially when moved onto the inner sleeve 20, and provides a radially inward force when positioned on the inner sleeve 20. For example, the fixation ring 30 transitions between a non-expanded diameter, when the fixation ring 30 is not provided on the inner sleeve 20, and an expanded diameter when the fixation ring 30 is provided on the inner sleeve 20, where the non-expanded diameter is less than the expanded diameter.

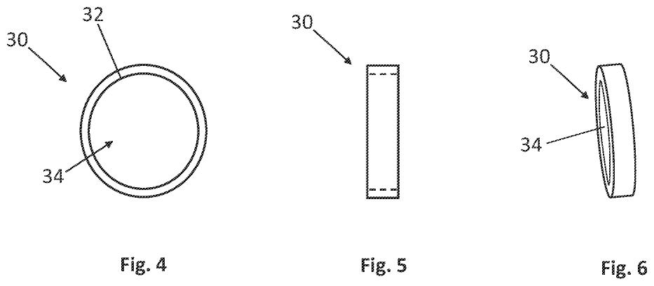

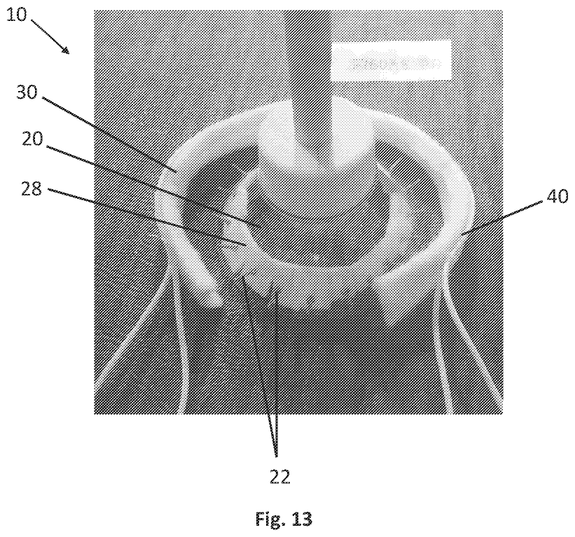

As illustrated in FIGS. 4-6, the fixation ring 30 defines a continuous cylindrical body portion. In other examples, such as shown in FIG. 13, the fixation ring 30 defines a discontinuous cylindrical body portion, e.g., a c-shaped fixation ring extending only partially around the circumference of the inner sleeve 20 such that the fixation ring 30 includes a gap extending through an entire thickness of the cylindrical body portion of the ring 30. A width of the gap increases when the fixation ring 30 is expanded for positioning over the inner sleeve 20. For example, when the fixation ring 30 is transitioned from a non-expanded configuration, when the fixation ring 30 is not provided on the inner sleeve 20, to an expanded configuration when the fixation ring 30 is provided on the larger diameter inner sleeve 20.

The axial length of the fixation ring 30 ranges from 2.0 mm to 45.0 mm, including exemplary values of 2 mm, 3 mm, 4 mm, 5 mm, 6 mm, 7 mm, 8 mm, 9 mm, 10 mm, 15 mm, 20 mm, 25 mm, 30 mm, 35 mm, 40 mm, 45 mm. In still further examples, the axial length of the fixation ring 30 can be between (and including) 2.0 mm to 10 mm, 10 mm to 20 mm, 30 mm to 45 mm. In some examples, the axial length of the fixation ring 30 corresponds with the axial length of the inner sleeve 20. The thickness of the fixation ring 30 ranges from 0.5 mm to 3.5 mm, including exemplary values of 0.5 mm, 1.0 mm, 1.5 mm, 2.0 mm, 2.5 mm, 3.0 mm, 3.5 mm. The inner diameter of the fixation ring 30 ranges from 3.5 mm to 33.5 mm, including exemplary values of 3.5 mm, 5 mm, 10 mm, 15 mm, 20 mm, 25 mm, 30 mm, 33.5 mm. The outer diameter of the fixation ring 30 rages from 4.0 mm to 37.0 mm, including exemplary values of 4.0 mm, 10 mm, 15 mm, 20 mm, 25 mm, 30 mm, 35 mm, 37.0 mm.

Though constructed to fit snugly around the inner sleeve 20 and patient's anatomy 60 (e.g., blood vessel), the fixation ring 30 can be constructed from a material that allows the fixation ring 30 it to be removed and/or repositioned over the inner sleeve 20. For example, as described below, the fixation ring 30 can be constructed from a polymer material, including an elastomeric polymer material, configured to stretch to a larger diameter and contract back towards the initial, unexpanded diameter. The fixation ring 30 can further include weakening structure, such as score lines and/or etchings, that allow the outer fixation ring 30 to tear or stretch along the weakened structure. The fixation ring 30 can include multiple weakening structures spaced around the circumference of the fixation ring 30. In use, the fixation ring 30 is positioned over the patient's anatomy 60 at a location corresponding to the inner sleeve 20. If the inner sleeve 20 and/or fixation ring 30 need to be repositioned or removed, the physician can tear or stretch the fixation ring 30 along the weakening structure/score line and remove the fixation ring 30 from the patient anatomy 60.

As illustrated in FIGS. 1A and 1B, the fixation device 10 further includes a ligature 40 extending around the inner sleeve 20 adjacent and/or within the groove 21. For example, where the groove 21 includes a V-shaped groove recessed in the outer surface 24 of the inner sleeve 20, the ligature 40 is positioned adjacent the groove 21 between the shoulder 27 and the tapered surface 29.

As illustrated in FIG. 1A, the ligature 40 secures the prosthetic device 50 to the inner sleeve 20. For example, a tubular-shaped prosthetic device 50 (e.g., arterial segment, a venous segment, an atrium structure, a graft material, a prosthetic heart valve, a heart assist pump, an artificial heart), is advanced over the distal end 23 of the inner sleeve 20. The ligature 40 is positioned around the outer surface of the prosthetic device 50. The ligature is tightened around the prosthetic device 50 such that the radially inward force provided by the ligature 40 creates a liquid-tight seal between the prosthetic device 50 and the inner sleeve 20, and the prosthetic device 50 is fixedly coupled to the inner sleeve 20.

In further examples, as shown in FIG. 1B, the ligature 40 is used to secure the fixation ring 30 and the prosthetic device 50 to the inner sleeve 20. For example, the ligature 40 is positioned around the outer surface of the fixation ring 30 securing the patient anatomy between the fixation ring 60 and the outer surface of the inner sleeve 20. The ligature 40 is tightened such that the radially inward force provided by the ligature 40 creates a liquid-tight seal between the inner sleeve 20, fixation ring 30, prosthetic device 50 and patient anatomy 60, ensuring hemostasis.

The ligature 40 is composed of biocompatible material. For example, the ligature 40 is composed of at least one of a woven or braided material (e.g., braided polyester, Dacron tape), a bead-style cable (e.g., a bead-style cable composed of nylon, polyethylene or polypropylene).

It is contemplated that the fixation device 10 includes fixation structure (i.e., inner sleeve 20 and fixation ring 30) coupled to opposing ends of the prosthetic device 50 so that the fixation device 10 can be used to join adjacent portions of patient anatomy, e.g., linking a prosthetic device between adjacent segments of a patient's blood vessel or other biological conduit.

For example, the fixation device 10 will include a first inner sleeve 20a and fixation ring 30a coupled to at the proximal end of the prosthetic device 50, and a second inner sleeve 20b and fixation ring 30b at the distal end of the prosthetic device 50. The second inner sleeve 20b and fixation ring 30b can include similar design and function as the first inner sleeve 20a and fixation ring 30a. Like the fixation structure at the proximal end of the prosthetic device 50, the second inner sleeve 20b and fixation ring 30b are sized and configured to receive a second portion of a patient's anatomy 60 (e.g., a second opening in the patient's blood vessel) therebetween such that the portion of a patient's anatomy 60 is fixedly secured between the second inner sleeve 20b and fixation ring 30b. As a result, the fixation device 10 couples adjacent arterial segments/adjacent biological conduit segments.

In general, the inner sleeve 20 and fixation ring 30 are constructed from a biologically inert material. For example, the inner sleeve 20 is constructed from at least one of a metal (e.g., stainless steel, nitinol, pyrolytic carbon, cobalt-chromium alloy (e.g., Stelite™) and a polymer (e.g., polyethylene, Teflon®, acetal homopolymer such as polyoxymethylene POM, Delrin™). In some examples, the fixation ring 30 is constructed from fabric tape or a felt tape (e.g., Dacron felt) and/or a felt material stiffened with glue. In some examples, the inner sleeve 20 is constructed from a magnetic material and/or includes magnetic elements that are magnetically attracted to the fixation ring 30. Alternatively, the fixation ring 30 can be constructed from a magnetic material and/or includes magnetic elements that are magnetically attracted to the inner sleeve 20.

The inner sleeve 20 includes a covering material (e.g., fabric) and/or coating on the outer surface 24 that prevents damage to the patient anatomy 60 and also provides for increased grip/resistance between the inner sleeve 20 and the patient's anatomy 40 when securing the fixation device 10.

Similarly, the inner sleeve 20 and/or fixation ring 30 can include a surface texture and/or coating to help secure the inner sleeve 20 and/or fixation ring 30 to the patient anatomy 60. In some examples, a mechanical and/or chemical fastener (e.g., an adhesive such as BioGlue™ by CryoLife) are used to secure the inner sleeve 20 and/or fixation ring 30 to the patient anatomy 60 (e.g., the corresponding inner/outer surface of the patient's blood vessel). In a further example fixation device 10, the inner surface 32 of the fixation ring 30 and/or outer surface of the inner sleeve 20 include a textured surface and/or coating for improving grip between the fixation device 10 and the patient anatomy 60. The textured surface and/or coating can also allow for tissue ingrowth between the fixation ring 30 and the patient's anatomy. It is contemplated that the outer surface 22 of the inner sleeve 20 can also include a textured surface and/or coating for improving grip with the patient anatomy 60 and/or allowing tissue ingrowth between the inner sleeve 20 and the patient anatomy 60. Example textured surfaces include a flocked surface, laser etched surface, a texture material deposited, an adhesive deposited on the surface of the inner sleeve 20 and/or fixation ring 30, and combinations thereof.

As described above and illustrated in FIGS. 1A and 1B, the prosthetic device 50 is coupled to incorporated into the inner sleeve 20. In other examples, the prosthetic device 50 is coupled to the fixation ring 30. In further examples, the prosthetic device 50 is separate from the inner sleeve 20 and fixation ring 30 and the fixation ring 30 and/or ligature 40 is used to secure the prosthetic device 50 to the inner sleeve 20.

As described above, example prosthetic devices 50 include a vascular graft material and/or other prosthetic biological conduit. The prosthetic device 50 is composed of a biocompatible synthetic material. Example biocompatible synthetic materials include polytetrafluoroethylene (PTFE), polyester (e.g., Dacron®, Gortex®), silk fibroin, polyurethane, and/or any other material known in the art that is suitable as a replacement for a biological conduit. In some examples, the prosthetic device 50 comprises at least one an arterial segment, a venous segment, an atrium structure, a prosthetic heart valve, a heart assist pump.

In some example anastomosis fixation devices 10, the prosthetic device 50 is impregnated with a material for promoting sealing and/or preventing infection. For example, the impregnation material can include a sealant for promoting sealing between the patient's vascular structure and the prosthetic device 50 (e.g., gelatin, collagen). Additionally/alternatively, the impregnation material can include an additive that inhibits bacterial infection (e.g., antibiotic, antiseptic). In certain examples, the prosthetic device 50 is a gelatin-impregnated woven polyester vascular graft.

The use of the example sutureless anastomotic fixation device 10 for aortic and other vascular surgeries is described below. As provided above, the use of the anastomotic fixation devices described herein allows for a rapid, hemostatic anastomotic techniques that will improve outcomes in complex operations such as the dissection of the ascending aorta and the resection of aortic aneurysms. The target patient anatomy 60 comprises a patient's blood vessel including, for example, an arterial segment, a venous segment, and/or an atrium structure. As described above, it is contemplated that the target patient anatomy 60 may include any other biological conduit such as a bile duct, ureter, or fallopian tube. The method for positioning the fixation device 10 within the patient anatomy 60 is described in reference to a patient blood vessel, however similar method may be used to connect a prosthetic device 50 to any other biological conduit.

An opening is first created in the patient's blood vessel, e.g., by transecting the patient's blood vessel. The diameter of the blood vessel is measured to identify an inner sleeve 20 and fixation ring 30 having a diameter corresponding to the measured diameter of the patient's blood vessel. The inner sleeve 20 is sized and configured to fit snugly within the patient's anatomy 60, e.g., circumferentially around the inner surface of a patient's blood vessel. Similarly, the fixation ring 30 is size configured to fit snugly on the outside of the patient's anatomy 60, e.g., circumferentially around the outer surface of the patient's blood vessel. In general and as will be described in more detail below, the pins 22 secure the location of the inner sleeve 20 with respect to the blood vessel and the fixation ring 30 provides a radially inward force for compressing the blood vessel between the fixation ring 30 and the inner sleeve 20, securing and creating a liquid-tight seal therebetween. A ligature 40 is used to provide a radially inward force prosthetic device 50 (and optionally the fixation ring 30) securing the prosthetic device 50 between the ligature 40 and the inner sleeve 20.

The fixation device 10 is coupled to the patient's anatomy by advancing the fixation device 10 to a treatment site at the opening in the patient's blood vessel. The proximal end of the inner sleeve 20 is advanced within the opening in the patient's blood vessel. The inner sleeve 20 is secured to the blood vessel by engagement between the pins 22 and the blood vessel. For example, the pins 22 can extend partially and/or completely through the wall thickness of the blood vessel.

The fixation ring 30 is then positioned adjacent an outer surface of the patient's blood vessel (adjacent the opening) at a location corresponding to the to the inner sleeve 20/pins 22. The blood vessel is positioned between the inner sleeve 20 and the fixation ring 30 around a circumference of the inner sleeve 20 such that at least one pin 22 extends through/into the blood vessel toward the fixation ring 30. The radial inward force provided by the fixation ring 30 creates a liquid-tight seal between the outer surface of the inner sleeve 20 and the blood vessel. As a result, a portion of the patient's blood vessel is secured between the inner surface 32 of the fixation ring 30 and an outer surface 24 of the inner sleeve 20.

In some examples, the distal ends of the pins 22 extend through/beyond the outer surface of the fixation ring 30. In this example, the portion of the pins 22 extending beyond the outer surface of the fixation ring 30 can be trimmed or otherwise shortened. A covering material can be provided over the fixation ring 30 and the trimmed ends of the pins 22.

As outlined above, the prosthetic device 50 is coupled to and/or incorporated with the inner sleeve 20. The prosthetic device 50 can be coupled to the inner sleeve 20 by advancing a tubular shaped prosthetic device 50 (e.g., arterial segment) on/over the distal end 23 of the inner sleeve 20. As such, the inner surface of the prosthetic device 50 is positioned adjacent the outer surface 24 of the inner sleeve 20.

A ligature 40 positioned such that the prosthetic device 50 is located between the inner sleeve 20 and the ligature 40. The ligature 40 is tightened around the outer circumference of the prosthetic device 50 and inner sleeve 20 such that the radially inward force provided by the ligature 40 creates a liquid tight seal between the outer surface 24 of the inner sleeve 20 and the prosthetic device 50. As a result, the prosthetic device 50 is fixedly secured to the inner sleeve 20. As described above, the outer surface 24 of the inner sleeve 20 includes a groove 21. In some examples, the ligature 40 is provided around the inner sleeve 20 and prosthetic device 50 at a location adjacent the groove 21 such that the ligature 40 is positioned between the shoulder 27 and the tapered surface 29.

In further examples, the ligature 40 is used to secure the fixation ring 30 to the inner sleeve 20. For example, the ligature is positioned around the outer surface of the fixation ring 30 and tightened, thereby securing the fixation ring 30 to the outer surface of the inner sleeve 20. The radially inward force provided by the ligature 40 creates a liquid-tight seal between the inner sleeve 20 and the fixation ring 30.

It is contemplated that the fixation device 10 can include a first ligature 40 securing the prosthetic device 50 to the inner sleeve 20 and a second ligature 40 securing the blood vessel between the fixation ring 30 and the inner sleeve 20.

In some examples, traction stitches are used to position the fixation device 10. For example, traction stitches are placed in the patient's blood vessel, e.g., single or multiple stitches placed at various circumferential positions around the blood vessel. Positioning the inner sleeve 20 and/or outer fixation ring 30 within the opening in the patient's blood vessel includes positioning or otherwise seating the traction stitch(es) adjacent the inner sleeve 20. For example, when the blood vessel comprises the aorta, the traction stitches are placed on the aorta to ensure the aorta is well seated into the inner sleeve 20. The traction stitches can be removed after the fixation device 10 has been secured to the patient's blood vessel.

In an example where the blood vessel comprises an aorta, positioning the inner sleeve 20 adjacent an outer surface of the patient's blood vessel includes positioning the inner sleeve 20 adjacent the adventitia of the aorta. The pins 22 are directed through the aorta tissue and the fixation ring 30 is positioned along the outer surface of the tissue adjacent the pins 22 securing the aorta tissue between the inner sleeve structure 20 and the fixation ring 30.

As described above, the fixation device 10 may include fixation structure (inner sleeve 20 and fixation ring 30) at both ends of the prosthetic device 50. This allows the fixation device 10 to be used to join adjacent portions of patient anatomy, i.e., linking the prosthetic device 50 between adjacent dissected segments of the patient's blood vessel or other biological conduit.

Accordingly, the fixation device 10 includes a first inner sleeve 20a and a first fixation ring 30a provided at the proximal end of the prosthetic device 50, and a second inner sleeve 20b and a second fixation ring 30b provided at the distal end of the prosthetic device 50. The fixation structure at the proximal end is coupled to the patient's blood vessel as described above. The fixation structure at the distal end of the prosthetic device 50 is then coupled to the patient's blood vessel by advancing the second inner sleeve 20b in the within a second opening in the patient's blood vessel. Pins 22 provided on the second inner sleeve 20b are passed through/into the patient's blood vessel and the second fixation ring 30b is positioned adjacent/around the outer surface of the patient's blood vessel at the location of the pins 22. The pins 22 secure the position of the inner sleeve 20 with respect to the blood vessel and the radially inward force provided by the fixation ring 30 against the outer surface of the blood vessel. As a result, a second portion of the patient's blood vessel is secured between the inner surface 32 of the fixation ring 30b and an outer surface 22 of the inner sleeve 20b.

In the example process, securing the portion of the patient's blood vessel between the fixation ring 30a, 30b and the inner sleeve 20a, 20b creates a liquid-tight seal between the inner sleeve 20a, 20b, the blood vessel, and the fixation ring 30a, 30b. The seal between the inner sleeve 20a, 20b, the blood vessel, and the fixation ring 30a, 30b is tested by flowing fluid (e.g., saline, blood) through the prosthetic device 50. For example, a clamp upstream of the prosthetic device 50 is released and blood is allowed to flow through the fixation device 10/prosthetic device 50. If a leak at the fixation device 10 is determined, i.e., a liquid-tight seal between the between the inner sleeve 20a, 20b, the blood vessel, and the fixation ring 30a, 30b is not present, the clamp is reapplied and the fixation ring 30a, 30b is removed and replaced, a ligature 40 is provided over the fixation ring 30a, 30b, and/or the fixation ring 30a, 30b and inner sleeve 20a, 20b are remove and replaced with different sized devices.

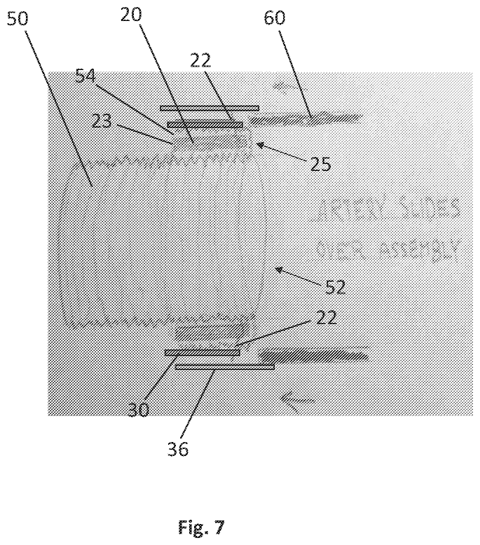

FIG. 7 illustrates a schematic drawing of patient with another example anastomosis fixation device 10. The fixation device 10 of FIG. 7 includes structure and materials similar to the fixation device 10 of FIGS. 1A-6. Like element numbers are used to identify like structure. The differences between the fixation device of FIGS. 1A-6 and the device of FIG. 7 are provided in more detail below.

The fixation device 10 of FIG. 7 includes an inner sleeve 20 sized and configured to be received within a blood vessel of a patient. The inner sleeve 20 includes pins 22 extending from the outer surface 24. As illustrated in FIG. 7, the pins 22 extend in a direction transverse to a longitudinal axis of the inner sleeve 20, e.g., the pins 22 are angled in a direction towards the distal end 23 of the inner sleeve 20. A tubular component, i.e., tubular-shaped prosthetic device 50, is provided within a central lumen 26 of the inner sleeve 20. As outlined above, the prosthetic devices 50 includes, for example, arterial segment, a venous segment, an atrium structure, a graft material, a vascular graft, a prosthetic heart valve, a heart assist pump, an artificial heart.

A portion 54 of the proximal end 52 of the prosthetic device 50 extends through and beyond the proximal end 25 and is folded over the proximal end 25 of the inner sleeve 20. The folded over portion 54 of the prosthetic device 50 extends along the outer surface 24 of the inner sleeve 20. Engagement between the folded over portion 54 and the pins 22 couples the prosthetic device 50/folded over portion 54 to the inner sleeve 20.

A fixation ring 30 is provided over the folded over portion 54 of the prosthetic device 50 and the pins 22 such that the folded over portion 54 is fixedly secured between the fixation ring and the inner sleeve 20. While the pins 22 secure the position of the prosthetic device 50 with respect to the inner sleeve 20, the radially inward force provided by the fixation ring 30 creates a liquid-tight seal between the inner sleeve 20 and the prosthetic device 50.

As illustrated in FIG. 7, the fixation device 10 includes a second fixation ring 36. The second fixation ring 36 is provided over the folded over portion 54 of the prosthetic device 50 and the pins 22. The patient anatomy 60 (e.g., atrial tissue) is received over the fixation ring 30 such that pins 22 extend into and/or through the patient anatomy 60 and the second fixation ring 60 is received over the pins 22. As such, the second fixation ring 36 is coupled to the fixation ring 30 and a portion of a patient's anatomy 40 is fixedly secured between an inner surface of the second fixation ring 36 and an outer surface of the fixation ring 30. The radially inward force provided by the second fixation ring creates a liquid-tight seal between the inner sleeve 20, fixation sleeve 30, second fixation sleeve 36 and the prosthetic device 50.

The use of the example sutureless anastomotic fixation device 10 of FIG. 7 is now described in reference to coupling an example prosthetic device 60 (e.g., artery graft material) to patient's blood vessel. An opening is first created in the patient's blood vessel, e.g., by transecting the patient's blood vessel. The diameter of the blood vessel is measured to identify an inner sleeve 20, fixation ring 30 and second fixation ring 36 having a diameter corresponding to the measured diameter of the patient's blood vessel. The inner sleeve 20, fixation ring 30 and second fixation ring 36 are selected such that the inner sleeve 20 fits snuggly around the prosthetic device 50 and the second fixation ring 36 fits snuggly over the blood vessel and fixation ring 30.

The prosthetic device 50 is coupled to the inner sleeve 20 by advancing a proximal end 52 of the prosthetic device 50 through the central lumen 26 of an inner sleeve 20. The prosthetic device 50 is advanced within the inner sleeve 20 until at least a portion of the proximal end 52 extends beyond a proximal end 25 of the inner sleeve 20. The portion of the prosthetic device 50 extending beyond the proximal end 25 of the inner sleeve 20 is folded over the proximal end 25 of the inner sleeve 20. The folded over portion 25 prosthetic device 50 extends along an outer surface 24 of the inner sleeve 20. The folded over portion 54 is coupled to the pins 22 projecting from the outer surface 24 of the inner sleeve 20 such that such that at least one pin 22 extends through/into the folded over portion 54 of the prosthetic device 50.

A fixation ring 30 is then positioned around the outer surface of the folded over portion 54 at a location corresponding to the to the inner sleeve 20/pins 22. The radial inward force provided by the fixation ring 30 creates a liquid-tight seal between the prosthetic device 50, inner sleeve 20 and the fixation ring 30.

The combined prosthetic device 50 and inner sleeve 20 is then coupled to the patience blood vessel by advancing the combined prosthetic device 50 and inner sleeve 20 into the blood vessel such that the outer surface of the fixation ring 30 is positioned adjacent an inner surface of the blood vessel. The blood vessel is secured to the prosthetic device 50 by engagement between the pins 22 and the blood vessel. For example, the pins 22 can extend partially and/or completely through the wall thickness of the blood vessel.

A second fixation ring 36 is then positioned adjacent the outer surface of the patient's blood vessel (adjacent the opening) at a location corresponding to the to the inner sleeve 20/pins 22. The blood vessel is positioned between the fixation ring 30 and the second fixation ring 36 around a circumference of the inner sleeve 20 such that at least one pin 22 extends through/into the blood vessel toward the second fixation ring 36. The radial inward force provided by the second fixation ring 36 creates a liquid-tight seal between the blood vessel and the prosthetic device 50, e.g., between the prosthetic device 50, inner sleeve 20, fixation ring 30, blood vessel and the second fixation ring 36. As a result, a portion of the patient's blood vessel is secured between the inner surface of the second fixation ring 36 and the outer surface of the fixation ring 30.

Similar to the fixation device 10 of FIGS. 1A-6, the fixation device 10 of FIG. 7 may include identical fixation structure (inner sleeve 20, fixation ring 30, second fixation ring 36) at both ends of the prosthetic device 50. This allows the fixation device 10 to be used to join adjacent portions of patient anatomy, i.e., linking the prosthetic device 50 between adjacent dissected segments of the patient's blood vessel/biological conduits.

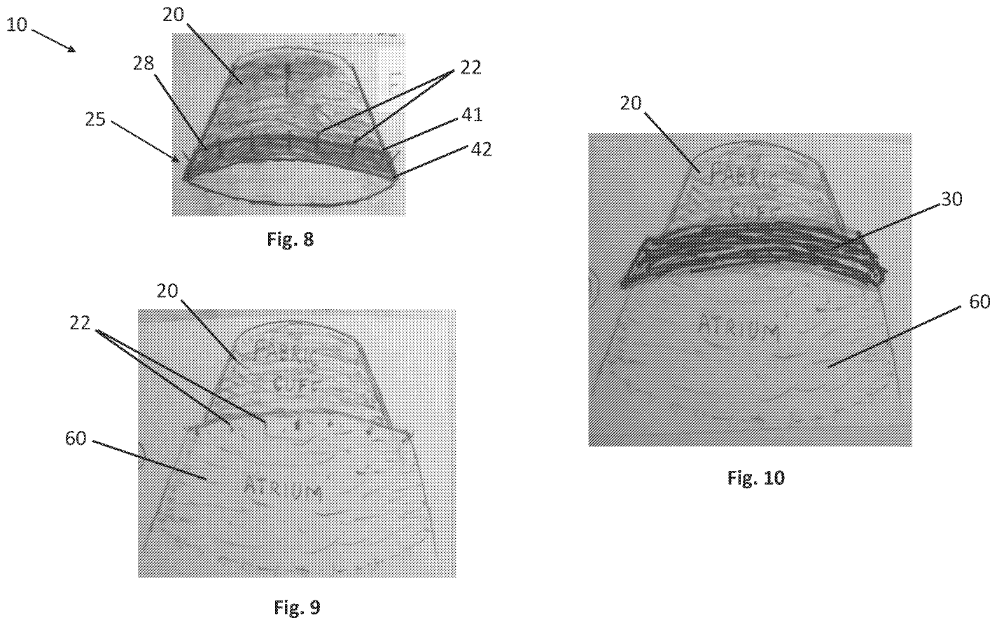

FIGS. 8-13 illustrate a schematic drawing of a prosthetic fixation device 10 according to another example. The fixation device 10 of FIG. 8 includes structure and materials similar to other fixation devices disclosed herein. Like element numbers are used to identify like structure. The differences between the fixation devices disclosed herein and the device of FIGS. 8-13 are provided in more detail below.

In this example, the fixation device 10 includes a flared sleeve 20, a circular band 28 coupled to a proximal end 25 of the flared sleeve 20. The circular band 28 includes pins 22 projecting radially outward from an outer surface of the band 28. A fixation ring 30 received over the band 28 such that the pins 22 extend at least partially into the fixation ring 30. The radially inward force provided by the fixation ring 30 creates a liquid-tight seal between the band 28 and the fixation ring 30, and any patient anatomy 60 (e.g., atrium) positioned therebetween. In this example, the prosthetic device 50 coupled to the flared sleeve 20 includes at least one of a prosthetic heart valve, a heart assist pump, an artificial heart. For example, the prosthetic device 50 is an atrial cuff of a total artificial heart.

As illustrated in FIG. 10, the band 28 has a shape corresponding to a conical segment. For example, the band 28 has a top edge 41 and a bottom edge 42, where the circumference defined by the top edge 41 is less than the circumference defined by the bottom edge 42. The band 28 is fixedly coupled to the proximal end 25 of the flared sleeve 20. In some examples, the band 28 is coupled to the flared sleeve 20 by a mechanical (e.g., suture) and/or chemical fastener (e.g., an adhesive such as BioGlue™ by CryoLife). The band 28 is constructed from a biologically inert material. For example, the band 28 is constructed from at least one of a metal (e.g., titanium, stainless steel, nitinol, pyrolytic carbon, cobalt-chromium alloy (e.g., Stelite™) and a polymer (e.g., polyethylene, Teflon®, acetal homopolymer such as polyoxymethylene POM, Delrin™). In some examples, the band 28 is constructed from fabric tape or a felt tape (e.g., Dacron felt) and/or a felt material stiffened with glue.

As with the fixation devices described here, in some examples, the pins 22 are equally spaced circumferentially around the outer surface of the band 28. In certain examples, the pins 22 are spaced circumferentially around the outer surface of the band 28 such that the spacing between adjacent pins 22 ranges from 4.5 mm to 5.5 mm, including exemplary values of 4.5 mm, 4.75 mm, 5.0 mm, 5.25 mm, 5.5 mm.

In some examples, the band 28 includes the number of pins ranges from 1 to 20 pins. In certain examples, the pins 22 extend from the outer surface of the band 28 a distance ranging from 2.0 mm to 4.0 mm, including exemplary values of 2.0 mm, 2.5 mm, 3.0 mm, 3.5 mm, 4.0 mm. As provided in FIGS. 8-12, the pins 22 extend from the band 28 at an angle towards the distal end 23 of the flared sleeve 20. In some examples, the pins 22 extend from the band 28 at an angle ranging from 250 to 350 relative to an axis extending perpendicular to a longitudinal centerline of the band 28.

As described above, the fixation device 10 includes a fixation ring 30 positioned circumferentially around the flared sleeve 20 with the patient anatomy 60 (e.g., atrium) therebetween. The pins 22 secure the position of the flared sleeve 20 with respect to the patient anatomy 60 and the radially inward force provided by the fixation ring 30 creates a liquid-tight seal between the flared sleeve 20/band 28, fixation ring 30 and patient anatomy 60. The fixation ring 30 can have a curved or rectilinear shape in cross-section. As illustrated in FIG. 13, the fixation ring 30 can have a U or C-shaped cross section, providing a portion for receiving the pins 22. In some examples, the fixation ring 30 defines a continuous cylindrical body portion extending around the circumference of the flared sleeve 20. In other examples, as provided in FIG. 13, the fixation ring 30 defines a discontinuous body portion and includes a gap extending through the thickness of the ring. The width of the gap increases when the fixation ring 30 is expanded for positioning over the flared sleeve 20.

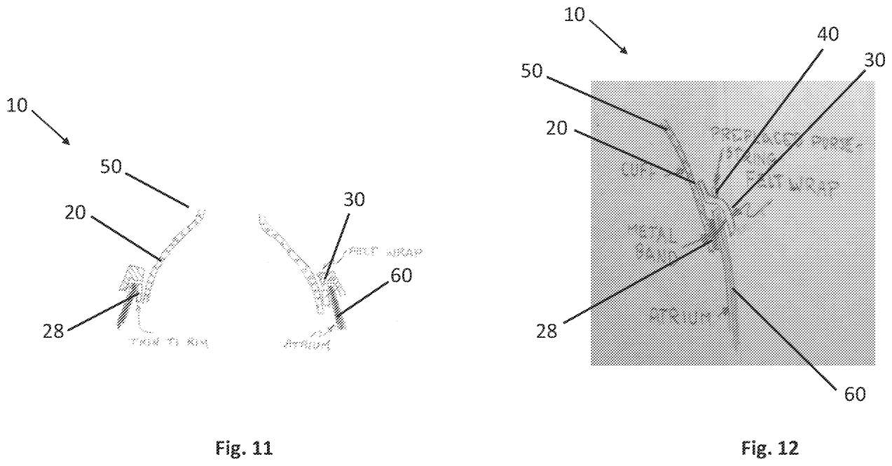

As illustrated in FIGS. 12-13, in some examples, the fixation device 10 includes a ligature 40 extending around the circumference of the fixation ring 30. Tightening the ligature around the fixation ring 30 creates a liquid-tight seal between the fixation ring 30 and the band 28.

The use of the example sutureless anastomotic fixation device 10 of FIGS. 8-13 is now described in reference to coupling an example prosthetic device 60 (e.g., artery graft material) to patient's blood vessel. In the present example, it is contemplated that a prosthetic device is coupled to the distal end 23 of the flared sleeve 20. An example prosthetic device 50 includes at least one of a prosthetic heart valve, a heart assist pump, an artificial heart. For example, the prosthetic device 50 is an atrial cuff of a total artificial heart.

An opening is first created in the patient's atrium and the open edges of the atrium are advanced over the proximal end 36 of the fixation device 10/flared sleeve 20. The atrium tissue is coupled to the flared sleeve 20 by engagement between the pins 22 and the tissue. For example, the pins 22 can extend partially and/or completely through the wall thickness of the atrium tissue. In some examples, the pins 22 are individually attached to the atrium tissue. In other examples, multiple pins 22 are simultaneously attached to the atrium tissue.

The fixation ring 30 is then positioned adjacent the outer surface of the atrium tissue at a location corresponding to the to the flared sleeve 20/band 28/pins 22. The atrium tissue is positioned between the flared sleeve 20 and the fixation ring 30 around a circumference of the flared sleeve 20 such that at least one pin 22 extends through/into the atrium tissue toward the fixation ring 30. The fixation ring 30 is secured around the flared sleeve 20/band 28, for example, by engagement between the pins 22 and the fixation sleeve 30. The radial inward force provided by the fixation ring 30 creates a liquid-tight seal between the outer surface of the flared sleeve 20 and the atrium tissue. As a result, a portion of the patient's atrium tissue is secured between the inner surface 32 of the fixation ring 30 and an outer surface 24 of the flared sleeve 20.

As outlined above, the fixation device 10 includes a ligature 40 extending around the fixation ring 30. The ligature 40 is tightened around the outer circumference of the fixation ring 30 such that the radially inward force provided by the ligature 40 creates a liquid tight seal between fixation ring 30, atrium tissue, and the flared sleeve 20.

In an example process, securing the portion of the patient's atrium between the fixation ring 30 and the flared sleeve 20 creates a liquid-tight seal between the flared sleeve 20, the atrium, and the fixation ring 30. The seal is tested by flowing fluid through the flared sleeve 20/prosthetic device 50. For example, an obturator is introduced into a central lumen of the atrial cuff. A foley catheter is advanced upstream of the prosthetic device and inflated to occlude blood flow through/from the pulmonary veins. Liquid (e.g., blood, saline solution) is provided into the central lumen of the atrial cuff and any leakage around the fixation ring 30 and the flared sleeve 20 is determined.

If a leak at the fixation device 10 is determined, i.e., there is not a liquid-tight seal between the flared sleeve 20, the atrium, and the fixation ring 30, the fixation ring 30 and possibly the flared sleeve 20 can be removed and repositioned and/or replaced by difference sized fixation ring 30/flared sleeve 20.

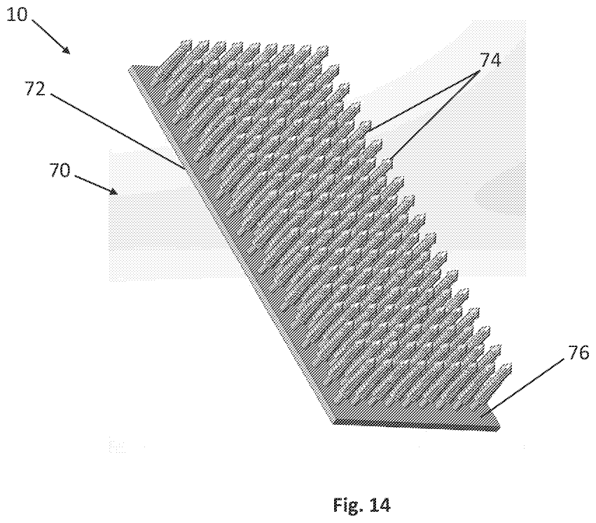

FIG. 14 illustrates a perspective side view of example anastomosis fixation device 10. The example fixation device 10 comprises a sheet 70 for joining adjacent tissue segments. The sheet 70 described herein can be used as the inner sleeve 20 and/or flared sleeve 20.

The example sheet 70 comprises a flexible substrate 72 including at a plurality of protrusions 74 extending from an inner surface 76 of the substrate 72. Alternatively, the protrusions 74 can extend from an outer surface of the substrate 72. In other examples, the protrusions 74 extend from both the inner and outer surfaces of the substrate 72.

In some examples, the protrusions 74 are similar in size, shape and function to the pins 22 of the fixation devices 10 described here. In particular, the protrusions 74 are sized and configured to anchor the substrate 72 to a tissue of a patient. In some examples, the protrusions 74 include an anchoring feature (e.g., a fish hook shape or barb at the distal end) for securing the protrusion within a patient's tissue and preventing the protrusion 74 from withdrawing from the anatomy.

In some examples, the flexible substrate 72 includes a plurality of openings extending between the inner surface 76 and the outer surface. The openings are sized and configured to receive a suture for coupling the flexible substrate 72 to the patient anatomy and/or the prosthetic device.

The flexible substrate 72 can define any regular or irregular shape. For example, the flexible substrate 72/sheet 70 can be provided as a sheet. In another example, the flexible substrate 72 can be shaped to define a structure/form that extends in three coordinate planes. For example, the substrate 72 can define a cylindrical/annular shape. In this example the protrusions 74 can extend from the inner and/or outer surface of the substrate 72.

In some examples, the sheet 70 is composed of a non-absorbable material. Example materials include including at least one of a metal (e.g., titanium, stainless steel, nitinol, pyrolytic carbon, cobalt-chromium alloy), a polymer (e.g., polyurethane), and combinations thereof. In another example, sheet 70 is composed of an absorbable material. It is contemplated that the flexible substrate 72 and the protrusions 74 are constructed from the same material. In further examples, the flexible substrate 72 and the protrusions 74 are constructed from different materials.

In some example anastomosis fixation devices 10, the sheet 70 is impregnated with a material for promoting sealing and/or preventing infection. For example, the impregnation material can include a sealant for promoting sealing between the patient's tissue and the sheet 70 (e.g., gelatin, collagen). Additionally/alternatively, the impregnation material can include an additive that inhibits bacterial infection (e.g., antibiotic, antiseptic).

The use of the example sutureless anastomotic fixation device 10/sheet 70 for connecting native blood vessels to vascular grafts and/or total artificial heart devices, or other biological conduit.

In some examples, the sheet 70 is used for joining adjacent tissue segments (e.g., adjacent arterial segments, venous segments, atrium structure). An opening is first created in the patient's blood vessel, e.g., by transecting the patient's blood vessel. The sheet 70/flexible substrate 72 is positioned adjacent a first tissue segment (e.g., a first blood vessel segment). The protrusions 74 are coupled to the first tissue segment by engagement between the protrusions 74 and the blood vessel. Next, the sheet 70/flexible substrate 72 is positioned adjacent a second tissue segment (e.g., a second blood vessel segment). The protrusions 74 are coupled to the second tissue segment by engagement between protrusions 74 and the second tissue segment.

Although several examples of the invention have been disclosed in the foregoing specification, it is understood by those skilled in the art that many modifications and other examples of the invention will come to mind to which the invention pertains, having the benefit of the teaching presented in the foregoing description and associated drawings. It is thus understood that the invention is not limited to the specific examples disclosed hereinabove and that many modifications and other examples are intended to be included within the scope of the appended claims. Moreover, although specific terms are employed herein, as well as in the claims which follow, they are used only in a generic and descriptive sense and not for the purposes of limiting the described invention nor the claims which follow. We, therefore, claim as our invention all that comes within the scope and spirit of these claims.

1. A prosthetic anastomosis fixation device comprising:
an inner sleeve sized and configured to be received within a blood vessel of a patient, the inner sleeve including a projection extending from an outer surface of the inner sleeve and a groove provide on the outer surface spaced apart from the projection;
a fixation ring removably received over the inner sleeve;
wherein engagement between the projection and the fixation ring couples the fixation ring to the inner sleeve.

2. The device of claim 1, wherein the projection includes pins extending from the outer surface of the inner sleeve in a direction transverse to a longitudinal axis of the inner sleeve.

3. (canceled)

4. The device of claim 2, where in the pins extends in a direction towards a distal end of the inner sleeve.

5. (canceled)

6. The device of claim 2, wherein a height of the pins is less than a wall thickness of the fixation ring such that the pins extend through a portion of a wall thickness of the fixation ring.

7. The device of claim 2, wherein a height of the pins is greater than a wall thickness of the fixation ring such that the pins extend through an entire wall thickness of the fixation ring.

8. (canceled)

9. (canceled)

10. (canceled)

11. The device of claim 1, wherein the projection is provided adjacent a proximal end of the inner sleeve and the groove is provided adjacent the distal end of the inner sleeve opposite from the projection,
wherein the groove includes V-shaped groove defined by a shoulder and a tapered surface extending from an inner edge of the shoulder toward the proximal end of the inner sleeve.

12. (canceled)

13. (canceled)

14. (canceled)

15. The device of claim 1, wherein the radially inward force provided by the fixation ring creates a liquid-tight seal between the inner sleeve and the fixation ring.

16. The device of claim 1, wherein the fixation ring has an inner diameter less than an outer diameter of the inner sleeve,
wherein the fixation ring transitions between a non-expanded diameter, when the fixation ring is not provided on the inner sleeve, and an expanded diameter when the fixation ring is provided on the inner sleeve, where the non-expanded diameter is less than the expanded diameter.

17. (canceled)

18. (canceled)

19. The device claim 1, wherein the fixation ring defines a discontinuous cylindrical body portion,
wherein the fixation ring includes a gap extending through an entire thickness of the cylindrical body portion,
wherein a width of the gap increases from a non-expanded configuration when the fixation ring is not provided on the inner sleeve to an expanded configuration when the fixation ring is provided on the inner sleeve.

20. (canceled)

21. (canceled)

22. (canceled)

23. The device of claim 1, wherein the fixation ring is constructed from a fabric tape or a felt tape.

24. The device of claim 1, further including a ligature extending around the inner sleeve adjacent the groove.

25. The device of claim 24, wherein the groove includes V-shaped groove defined by a shoulder and a tapered surface extending from an inner edge of the shoulder toward the proximal end of the inner sleeve,
wherein the ligature is provided adjacent the groove between the shoulder and the tapered surface.

26. (canceled)

27. The device of claim 1, wherein the inner sleeve is coupled to a prosthetic device, wherein the prosthetic device comprises at least one an arterial segment, a venous segment, an atrium structure, a graft material, a prosthetic heart valve, a heart assist pump, an artificial heart.

28. (canceled)

29. A method of attaching a prosthetic device to a patient's blood vessel, the method comprising:
advancing the proximal end of an inner sleeve within a blood vessel of a patient, the inner sleeve including pins extending from an outer surface of the inner sleeve;
securing the inner sleeve to the blood vessel by engagement between the pins and the blood vessel;
providing a fixation ring around the inner sleeve such that the blood vessel is positioned between the inner sleeve and the fixation ring around a circumference of the inner sleeve, and at least one pin extends through the blood vessel toward the fixation ring;
advancing a second tubular component on the distal end of the inner sleeve;
providing a ligature around the inner sleeve such that the second tubular component is provided between the inner sleeve and the ligature around the circumference of the inner sleeve; tightening the ligature around the inner sleeve and the second tubular component,
wherein the radial inward force provided by the fixation ring creates a liquid-tight seal between the outer surface of the inner sleeve and the blood vessel,
wherein tightening the ligature around the inner sleeve and the second tubular component creates a liquid-tight seal between the outer surface of the inner sleeve and the second tubular component.

30. The method of claim 29, wherein the second tubular component comprises the prosthetic device including at least one of arterial segment, a venous segment, an atrium structure, a graft material, a prosthetic heart valve, a heart assist pump, an artificial heart.

31. The method of claim 29, wherein the inner sleeve includes a groove on its outer surface,
wherein providing a ligature around the inner sleeve and the second tubular component includes positioning the ligature over the inner sleeve adjacent the groove.

32. The method of claim 31, wherein the groove includes V-shaped groove defined by a shoulder and a tapered surface extending from an inner edge of the shoulder toward the proximal end of the inner sleeve,
wherein providing a ligature around the inner sleeve and the second tubular component includes positioning the ligature between the shoulder and the tapered surface.

33. The method of claim 29, further including trimming a portion of the pins extending through the fixation ring.

34. A prosthetic fixation device comprising:
an inner sleeve sized and configured to be received within a blood vessel of a patient, the inner sleeve including pins extending from an outer surface of the inner sleeve;
a tubular component provided within a central lumen of the inner sleeve, a proximal end of the tubular component extending beyond a proximal end of the inner sleeve and a portion of the tubular component folded over the proximal end of the inner sleeve;
wherein engagement between the folded over portion of the tubular component and the pins couples the tubular component to the inner sleeve.

35. The device of claim 34, wherein the tubular component comprises at least one of a vascular graft, an arterial segment, a venous segment, an atrium structure, a graft material, a prosthetic heart valve, a heart assist pump, or an artificial heart.

36. (canceled)

37. (canceled)

38. (canceled)

39. (canceled)

40. (canceled)

41. The device of claim 34, further including a second fixation ring provided over the folded over portion of the tubular component and the pins,
wherein the radially inward force provided by the second fixation ring creates a liquid-tight seal between the inner sleeve, fixation sleeve, second fixation sleeve and the tubular component.

42-73. (canceled)
